(19) United States
(12) Patent Application Publication
(10) Pub. No.: US 2019/0105652 A1
(43) Pub. Date: Apr. 11, 2019

(54) SYSTEM AND METHOD FOR ADHERENCE MARKER DETECTION

(71) Applicant: MEDTECH BIOSCIENCE LLC, Buffalo Grove, IL (US)

(72) Inventors: William B. LESLIE, Chicago, IL (US); Jameson EMLING, Cary, IL (US)

(21) Appl. No.: 15/543,378

(22) PCT Filed: Sep. 24, 2015
(86) PCT No.: PCT/US2015/051848

§ 371 Date: Jul. 13, 2017

Related U.S. Application Data
(60) Provisional application No. 62/054,696, filed on Sep. 24, 2014.

(51) Int. Cl. B01L 3/00; G01N 33/543; G01N 33/53; G01N 33/208; B01J 31/26

(52) U.S. Cl. CPC B01L 3/502; G01N 33/54366; G01N 33/5302; G01N 33/54346; G01N 2600/00; B01J 31/26; B01L 2400/0605; B01L 2400/0638; G01N 33/208

(57) ABSTRACT

The present invention provides a system and method for detecting a target in a sample. In one aspect, a collection device includes a collection chamber, a test chamber, a passageway between the collection chamber and the test chamber, and a detection system positioned within the test chamber. The detection system includes at least one of a spiropyran, a molecularly imprinted polymer and a nonimprinted polymer.

### CROSS-REFERENCE TO RELATED APPLICATIONS

Not applicable.

### STATEMENT REGARDING FEDERALLY SPONSORED RESEARCH

Not applicable.

### BACKGROUND OF THE INVENTION

The present invention generally relates to colorimetric detection. In particular, the invention is related to compositions, devices and methods related to spiropyran-based colorimetric readout detection using molecularly imprinted polymers (MIPs), nonimprinted polymers (NIPs), polymer solutions, or spiropyran solutions.

Drug testing is a procedure which is carried out for a variety of reasons. In one aspect, a urine sample may be collected in a collection container. The sample may be subjected to a number of chemical or physical tests to detect the presence or absence of one or more unique drug targets within the sample.

In one example, a urine sample collection device can include two or more distinct chambers in which a first chamber of large volume receives and stores a sample of appreciable volume and a second chamber has therein a test card supporting chemistry for tests for an array of drugs. The urine sample may flow automatically between the chambers or a technician may manipulate the device to transfer a portion of the urine sample from the storage chamber to the test chamber where it reacts with prepared chemistry therein to produce a visual result. However, depending on factors such as the design of the device and the chemistry employed, such a device may be expensive or inaccurate. Accordingly, there is a need for a system and method for detection of one or more targets (adherence markers) in a sample.

### SUMMARY OF THE INVENTION

The present invention overcomes the aforementioned drawbacks by providing a system and method for the detection of adherence markers in a sample.

In accordance with one aspect of the present disclosure, a collection device includes a collection chamber, a test chamber, a passageway between the collection chamber and the test chamber, and a detection system positioned within the test chamber. The detection system includes at least one of a spiropyran, a molecularly imprinted polymer and a nonimprinted polymer.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIGS. 1A-IC show an embodiment of a collection device according to the present disclosure. FIG. 1A is an elevational view of a collection device. FIG. 1B is a perspective view thereof in which the cap has been removed from the cup. FIG. 1C is a cross-sectional view as taken along line IC-IC of FIG. 1A showing a collection chamber and a separate test chamber of the collection device.

FIGS. 2A-2B shows a photographic image of spiropyran solutions with or without quinine. A control solution without quinine is shown on the far left and three test solutions containing quinine are shown on the right. In the presence of quinine, the solution turns from yellow to red. FIG. 1A illustrates test samples including 6 mg of quinine in 0.5 mL spiropyran solution. FIG. 1B illustrates test samples including 6 mg of quinine in 1.0 mL spiropyran solution. A color change from red to orange/red is observed as quinine concentration is lowered.

FIG. 2C-2D show absorbance data for spiropyran solutions with or without quinine. FIG. 2A shows a photographic image of spiropyran solutions without (left) or with (right) quinine. In the presence of quinine, the solution turns from yellow to red. FIG. 2B shows absorbance spectra collected for the samples shown in FIG. 2A. A noted increase in absorbance at about 525 nm is observed for test samples including quinine.

FIG. 3 shows a photographic image of spiropyran solutions with or without quinine across a range of concentrations from 0 mg of quinine per 1 mL to 10 mg of quinine per 1 mL spiropyran solution. Color shifts from yellow to a red as quinine the concentration increases.

FIG. 4 shows absorbance spectra collected for the samples shown in FIG. 3. A noted increase in absorbance at about 525 nm is observed for test samples including quinine.

FIG. 5A-5B show photographic images of polymer solutions with or without quinine. FIG. 5A illustrates the polymer solution control sample as yellow (left), and the test sample as red in the presence of quinine (right), at a concentration of 6 mg per 0.5 mL. FIG. 5B illustrates the polymer solution as control (left) and test sample (right). The test sample in 5B is at a concentration of 6 mg per 1 mL polymer solution.

FIG. 6A-6B show absorbance data for polymer solutions with or without quinine. FIG. 6A shows a photographic image of polymer solutions without (left) or with (right) quinine. In the presence of quinine, the solution turns from yellow to red. FIG. 6B shows absorbance spectra collected for the samples shown in FIG. 6A. A noted increase in absorbance at about 525 nm is observed for test samples including quinine.

FIG. 7 shows solidified polymer materials in absence of quinine (top), and in the presence of quinine (bottom).

### DETAILED DESCRIPTION OF THE INVENTION

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by any later-filed nonprovisional applications.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. The terms “comprising” and variations thereof do not have a limiting meaning where these terms appear in the description and claims. Accordingly, the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

As used herein, the term “analyte” refers to a substance, atom or molecule of interest, such as a chemical, which may be detected by a color readout system.

As used herein, the term “color system” refers to a solution phase or a solid phase of a Polymer, or a SP solution, which changes color in the presence of an analyte.

As used herein, the term “chromophore” refers to a molecule, which undergoes conformation or isomerization change under a condition, leading to a visible color change of the solution having the molecule.

As used herein, the term “polymer or hydrogel solution” refers to a solution containing at least some of the polymer or hydrogel components, which is soluble in the solution. It may include both MIP and NIP color systems.

As used herein, the terms “polymer” and “hydrogel”, referring to the same or similar meaning in the invention, are often interchangeable throughout the invention. The terms “polymer” and “hydrogel” include both solid phases of polymers or hydrogels and polymer or hydrogel solutions.

As used herein, the terms “imprinting molecule” and “analyte”, referring to the same or similar meaning in the invention, are interchangeable throughout the invention unless indicated otherwise.

As used herein, the terms “colorimetric readout” and “colorimetric detection”, referring to the same or similar meaning in the invention, are interchangeable throughout the invention unless indicated otherwise.

As used herein, smart polymers, stimuli-responsive polymers or stimuli-responsive hydrogels are high-performance polymers or hydrogels that change according to the environment they are in. Such materials can be sensitive to a number of factors, such as temperature, humidity, pH, the intensity of light or an electrical or magnetic field and can respond in various ways, like altering color or transparency, becoming conductive or permeable to water or changing shape (shape memory polymers). Usually, slight changes in the environment are sufficient to induce greater change in the polymer's properties.

Smart polymers and hydrogels may appear in highly specialized applications and everyday products alike. They may be used for the production of hydrogels, biodegradable packaging, and to a great extent in biomedical engineering. One example is a polymer that undergoes conformational change in response to pH change, which may be used in drug delivery. In one embodiment, the present invention relates to smart polymers, stimuli-responsive polymers or stimuli-responsive hydrogels for colorimetric detection.

As used herein, the term “kit”, refers to a device for detection of an analyte in a sample by colorimetric readout. Specifically, in the present invention, a kit for detection of an analyte in a sample by colorimetric readout may comprise any composition as discussed in the specification, a solid support, and a means for detecting a visible color change of the composition, thereby providing detection of the analyte by colorimetric readout.

Molecularly imprinted polymers (MIPs) are the polymers which have been processed by a molecular imprinting technique, and these polymers thus possess cavities in polymer matrix with affinity to a chosen template molecule (see, for example, International Patent Application Publication No. PCT/US2013/065415 to Knop et al.). A molecular imprinting technique is a laboratory technique commonly adapted by many scientists in the field. Briefly, molecular imprinting is a technique that creates specific recognition sites for a target molecule within a synthetic polymer, and the goal of a molecular imprinting technique is to make an artificial lock for a specific molecule which serves as the key.

A general formulation and procedure to make molecularly imprinted polymers may include a cross-linking monomer and a template (the imprint molecule). Functional and cross-linking monomers are copolymerized in the presence of a template (the imprint molecule) in a suitable solvent. The template may be the target molecule or any structural derivatives of the target molecule. MIPs show specific binding to the imprint molecules.

After polymerization and subsequent removal of the imprinted molecules, cavities are produced within the polymer matrix showing specific sizes, shapes, and chemical functionalities complementary to those of the template molecules. Consequently, the resulting MIPs show specific affinity with the template molecules (the imprint molecules). The resulting MIP contains recognition sites, with shape and functional groups complementary to the imprint molecule. By using the above molecular imprinting technique, a molecular memory is introduced into the resulting MIPs, which is capable of selectively binding specific target molecules. Thus, the techniques and the MIPs may be used to fabricate sensors with heightened sensitivity and selectivity.

The indicator mechanism of the present invention may be operated via any suitable process resulting in a permanent and detectable change in the template or in the indicator (sensor) mechanism. For example, a permanent and detectable change may be a color change caused by the shift of UV-Vis absorption bands of the indicator compounds under certain conditions. In one embodiment, the present invention is about a SP solution, which changes colors upon addition of a chemical or an analyte.

In some embodiments, detection of an analyte may involve photochromism or solvatochromism. Photochromism is the reversible transformation of a chemical species between two forms by the absorption of electromagnetic irradiation, where the two forms have different absorption spectra. Photochromism usually refers to compounds that undergo a photochemical reaction where an absorption band in visible region of the electromagnetic spectrum changes dramatically in strength or wavelength. Solvatochromism refers to the ability of a chemical substance to change color due to a change in solvent polarity.

One example of photochromism includes the isomerization of spiropyran to merocyanine. Upon change of environments such as UV light irradiation, solvent polarity, pH, or chemicals, the isomerization takes place where the bond between the oxygen of the benzopyran and the spiro-carbon breaks, leading to the opening of the pyran ring. The resulting form of merocyanine possesses a highly conjugated double-bond system, with a capability of absorbing photons in the visible light region of the electromagnetic spectrum. Therefore, merocyanine appears to be colored.

In one embodiment of the present invention, photochromism of spiropyran and merocyanine isomerization may be used as the indicator mechanism of color readout. It is found that the interaction of certain monomers and molecules with the two forms of SP or MC may drive the equilibrium towards the colorless or colored states, forming the fundamentals of optical readout system in the present invention. It is believed by some researchers that different MC isomers yield different colors. Thus. photochromism of SP and MC isomerization may enable a color readout system showing different colors.

In one embodiment, the invention is a non-imprinted polymer (NIP) capable of color readout. In one aspect, there are no specific binding sites forming in the process of the NIP. Thus, unlike MIPs, NIPs do not possess specific binding ability to a pre-designed analyte or chemical. However, NIPs may be useful under a simple environment where specificity is not required and a fast result is highly expected. These types of environments may be seen mostly in production settings where the molecule to be detected is very specific and delays in this detection could be very costly, thus making the rapid detection a top priority. Field testing would be another beneficial application of the product as some companies require validation testing before importation. While the NIP lacks specificity, it does still offer a certain level of retention.

In one embodiment, an MIP or NIP system may be incorporated into a split cup type design for the detection of a target in a sample. Examples of targets can include an illicit substance, a drug, an adherence marker or a metabolite. Some targets that may be detected with a system and method according to the present disclosure are described, for example, in U.S. patent application Ser. No. 14/190,960 to Knop et al.

In general, one aspect of the present disclosure includes detection of an adherence marker. Medication adherence usually refers to whether patients take their medications as prescribed, as well as whether they continue to take a prescribed medication. It has been observed that medication nonadherence is prevalent and may be associated with adverse outcomes and higher costs of care. There are many different methods for assessing adherence to medications including direct and indirect methods. Direct methods can include measurement of the level of medicine a metabolite or an adherence marker (e.g., a marker co-administered with a prescription drug) in a sample such as a blood or urine sample.

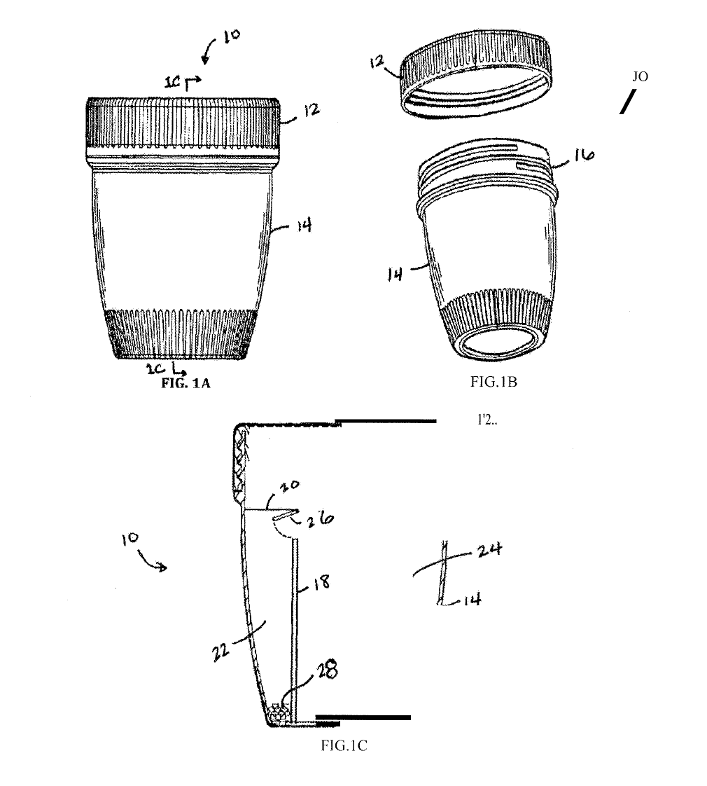

In one embodiment of the present disclosure, an adherence marker or other target molecule may be detected with a split chamber collection device. With reference to FIGS. 1A-1C, a collection device 10 can include a cap 12 that may be coupled to a collection cup 14 for collecting a sample such as a urine sample. The cup 14 can include a threaded upper end 16 or another suitable fastening mechanism for reversibly (or irreversibly) coupling the cap 12 to the cup 14. In on aspect, the cup 14 can include one or more internal partition walls such as a generally vertical partition wall 18 and a generally horizontal partition wall 20. Together, partition walls 18 and 20 may define a distinct test chamber 22 that may be in fluid communication with a collection chamber 24 by way of a swinging door 26 or another passageway.

In one aspect, the door 26 may be a two-way design where a sample or other material may freely pass between the collection chamber 24 and the test chamber 22. In another aspect, the door 26 may be a one-way design where a sample or other material may only pass from collection chamber 24 and the test chamber 22. In still another aspect, the door 26 may be configured to meter a specific amount of a sample or other material from the collection chamber 24 to the test chamber 22. In a further aspect, the door 26 may be configured to retain a detection system 28.

In some embodiments, the collection device 10 may include a detection system 28 for detecting a target. In one aspect the detection system 28 can be a liquid solution or a solid material such as a solid polymer or a hydrogel material. Example detection systems can include MIP or NIP systems as described herein. For example a detection system may be a solution including spiropyran, acrylic acid and water. The solution may be used to detect an adherence marker such as quinine. In one aspect, the quinine may be co-administered to a patient with a drug in order to determine if the patient has been properly adhering to the prescription dosage. The color of the spiropyran solution may be observed to change color, such as from yellow to red in the presence of quinine. Accordingly, if a urine sample is collected from a patient that has adhered to the prescription dosage of the drug, when the urine sample comes into contact with the spiropyran solution, the detection system may change from yellow to red. However, if the patient has not adhered to the prescription dosage, then no color change may be observed. Other suitable detection systems may be used such as solid polymer or polymer solution systems as will be described in the following Examples.

In operation, a patient may be provided with a collection device 10 including a detection system 28 retained in the test chamber 22. A patient may remove the cap 12 from the cup 14 and a urine sample may be collected in the collection chamber 24 of the cup 14. Thereafter, the patient may couple the cap 12 to the cup 14 and return the collection device 10 to a technician. The technician may then manipulate the device 10 to actuate the door 26 in order to transfer the urine sample from the collection chamber 22 the test chamber 22 where the sample may come into contact (e.g., mix) with the detection system 28. Thereafter, the technician may observe a color change in the detection system or another indication of the presence or absence of a target in the urine sample.

### Example 1—Solution Preparation

For the preparation of spiropyran solutions, 3 mg of spiropyran was dissolved in 685 flL of acyclic acid and sonicated for approximately 15 minutes. Next, 20 mL of deionized water or phosphate buffered saline solution was combined with the sonicated spiropyran in acrylic acid and the resulting mixture was inverted until fully mixed and then sonicated for approximately 2 minutes.

For the preparation of molecularly imprinted polymer solutions, 3 mg of spiropyran was dissolved in 685 μl, of acyclic acid and sonicated for approximately 10 minutes. Next, 3.556 g acrylamide, 0.0077 g N,N-methylene bisacrylamide and 0.0035 g 4,4-azobis(4-cyanovaleric acid) were combined in a 20 mL glass vial. The components in the glass vial were then combined with 20 mL of spiropyran solution in phosphate buffered saline as described above. The resulting mixture was inverted until fully mixed and then sonicated to provide a solution having a yellow-gold color.

Solid polymer materials were prepared by first combining 1.37 mL acrylic acid. 0.16 g sodium hydroxide, 20 flL polyethylene glycol diacrylate (PEGDA) and 40 mg 4,4-azobis(4-cyanovaleric acid) in a 20 mL glass vial. The components were then shaken and sonicated. Next, the sonicated components were combined with 20 mL of spiropyran solution in phosphate buffered saline as described above. The resulting mixture was inverted until fully mixed and then sonicated to provide a solid polymer solution.

### Example 2—Detection of Quinine with Spiropyran Solutions

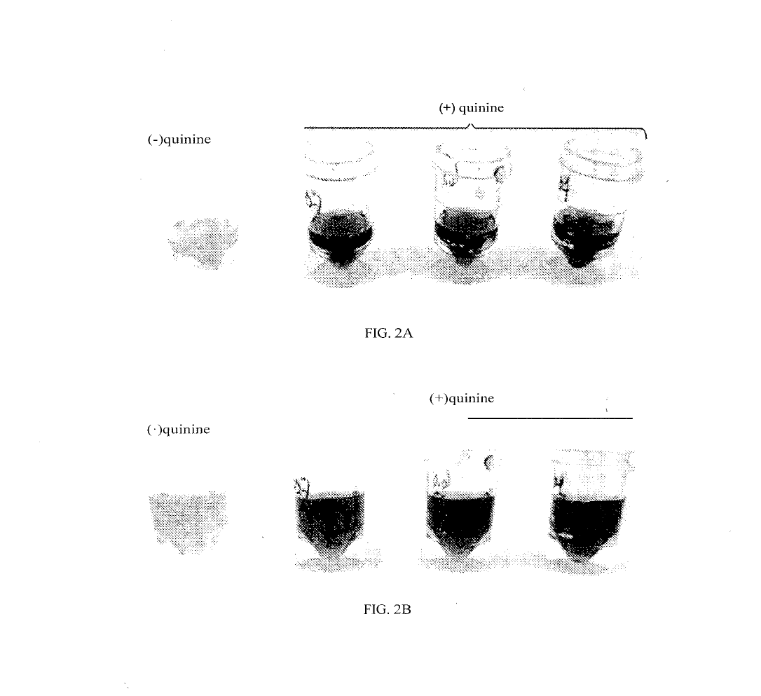

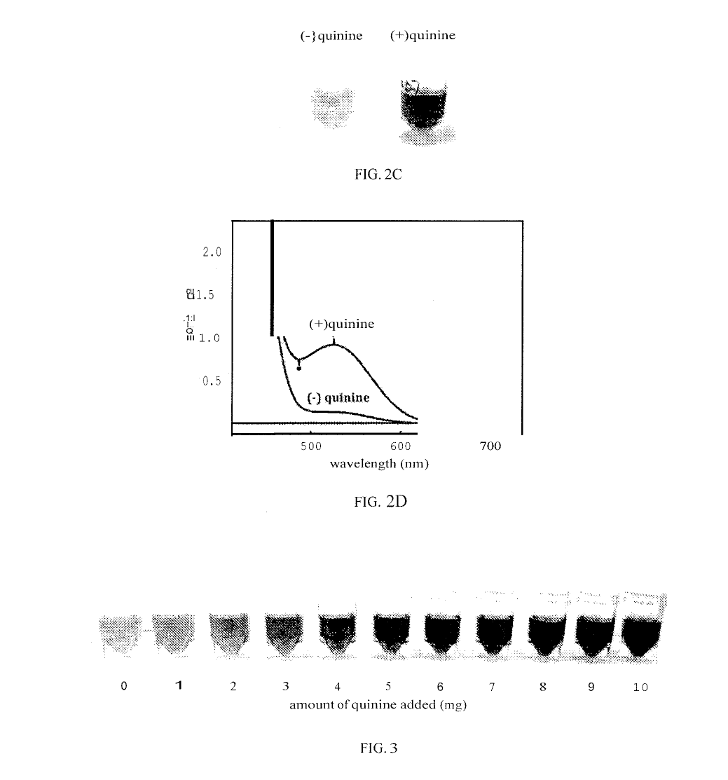

Spiropyran solution was distributed into polystyrene cups to provide control and test samples. Control samples included 1 mL of spiropyran solution and test samples includes 1 mL of spiropyran solution combined with 6 mg of quinine. As shown in FIGS. 2A-2B, a color change from yellow to red was observed upon the addition of quinine to the spiropyran solution. By comparison, the control samples remained yellow.

UV-Vis data was observed to corroborate the qualitative change in color of the spiropyran solution upon the addition of quinine. With reference to FIGS. 2C-2D, there was an increase in absorbance at about 525 nm for a spiropyran solution including Quinine as compared with a control solution. The change in the absorbance spectrum was consistent with a color change driven by the isomerization of spiropyran to merocyanine. This data corresponded with the proposed isomers associated with the “chromic” properties of spiropyran.

In another example, a quinine color scale was created with spiropyran solution. The concentration of quinine was increased in the spiropyran solution at 1 mg per mL increments as seen in FIG. 3. A concentration dependent color change from yellow to red as was observed as the quinine concentration was increased in the spiropyran solution.

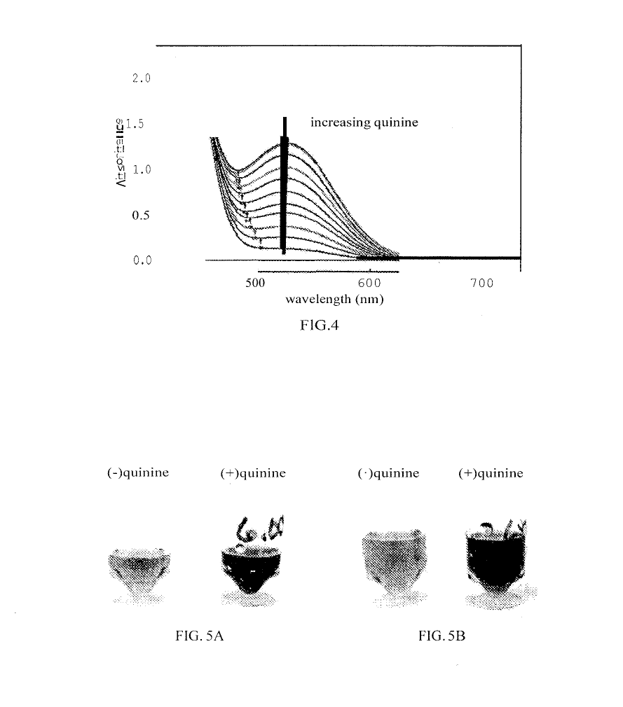

UV-Vis absorbance spectra were also collected for each of the samples in the prepared color scale as shown in FIG. 4. An increase in the absorbance peak at about 525 nm was correlated with increasing quinine concentration. The data was consistent with the isomerization of spiropyran to merocyanine.

### Example 3—Detection of Quinine with Polymer Solutions

Polymer solution testing was performed with a similar procedure as described above for spiropyran solutions. As shown in FIGS. 5A-5B, a color change from yellow to red was observed upon addition of quinine to the polymer solution. In one aspect, polymer solutions appeared to have a darker red color as compared with the orange-red color observed experiments with spiropyran solution.

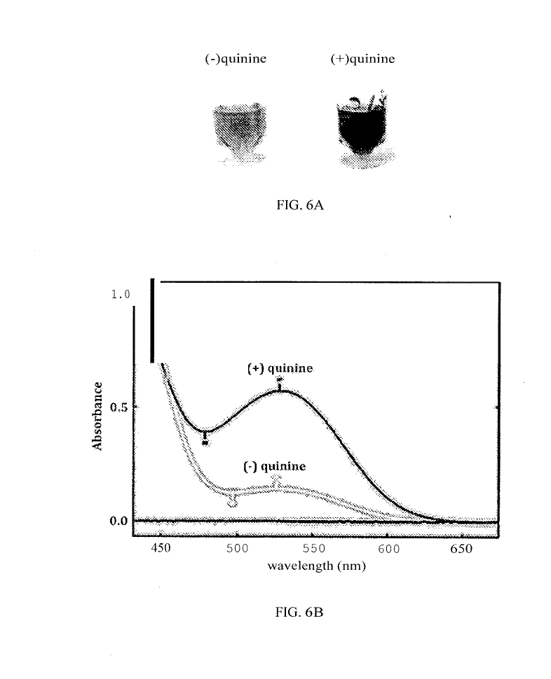

UV-VIS absorbance spectra were collected to provide a quantitative analysis of the color changes observed for polymer solutions in the presence of quinine (FIGS. 6A-6B).

### Example 4—Detection of Quinine with Solidified Materials

Solid polymer samples were tested as a platform for detection of quinine. The solid polymer platform differed from the spiropyran solution and polymer solution testing described above.

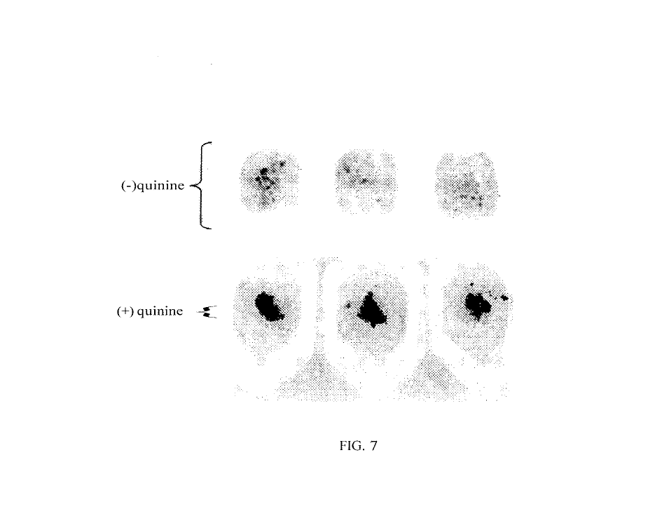

In one aspect, solid polymer samples were prepared by exposing 2 mL of solid polymers solution in a weigh boat to UV light to provide a solidified material. The solidified material was then cut into small pieces and incubated in the presence of spiropyran solution for 1 hour in order to diffuse spiropyran solution into the solidified material. Control samples were prepared by combining the incubated solidified material with 45 uL acrylic acid and 20 mL phosphate buffered saline. Test samples were prepared by combining the incubated solidified material with 200 mg quinine, 45 uL, acrylic acid and 20 mL phosphate buffered saline. The control samples were observed to remain a yellow color whereas the test samples changes from yellow to red upon exposure to quinine as shown in FIG. 7.

Detection of acetazolamide at low concentrations is accomplished through a layer-by-layer assembly of gold nanoparticles having carbonic anhydrase (CA) molecules on their surface and water soluble polymers with pendant groups bearing moieties of CA inhibitors. This could produce a system with optical properties that can be controlled by the attachment of multiple nanoparticle layers. This also allows controlling the intensity of the color observed. At the same time, the proximity of the nanoparticles to each other when binding the polymer layers could produce a shift in the light absorption properties of the system, thus changing the color of the system. Such color changes usually range from blue or purple when particles are in close proximity to each other, to red when the particles are separated from each other. The mode of detection thus consists on using a CA inhibitor moiety on the polymer with lower binding affinity than acetazolamide. In this manner, the nanoparticles-polymer system is assembled in the absence of acetazolamide. When acetazolamide is introduced in the system, its higher affinity will compete for the binding sites on CA and this can produce a separation of the nanoparticles from the polymer. At low concentrations of acetazolamide, this produces a noticeable color change as the nanoparticles become separated from each other. At large enough concentration, the entire assembly of nanoparticles and polymers are totally disrupted, resulting in the loss of color when the assembly is disintegrated.

Applicants previously demonstrated a method of analyte detection using gold (Au) nanoparticles. In one embodiment of the invention, it is found that the present MIP and NIP systems may be applied as color readout systems in the present of Au nanoparticles. Color changes of both MIP and NIP systems in the presence of Au nanoparticles. The polymer or hydrogel solutions of an MIP system and NIP systems in the absence of chromophore (SP and MC)

Applicants previously demonstrated a method of analyte detection using gold (Au) nanoparticles. In one embodiment of the invention, it is found that the present MIP and NIP systems may be applied as color readout systems in the presence of Au nanoparticles. The color changes of both MIP and NIP systems in the presence of Au nanoparticles. The polymer or hydrogel solutions of an MIP system and NIP systems in the absence of chromophore (SP and MC] were made. Even in the absence of a chromophore, the interaction between the analyte of disodium succinate and Au nanoparticles led to a red color of the resulting solution for the MIP system. The NIP systems, having no analyte of disodium succinate, exhibit a different color of purple-black. Thus, a color readout system may be created using Au nanoparticles instead of the chromophore of SP and MC. Further, color changes of both MIP and NIP systems in the presence of Au nanoparticles and the chromophore (SP and MCI. The polymer or hydrogel solid forms in solutions for the MIP system illustrates a red color, while the solutions for the NIP systems appear to be brown. Thus, Au nanoparticles have an effect the NIP systems by changing the color from yellow-orange to brown. The polymers here are representative of the initial time period of solution with buffer and disodium succinate solutions being added respectively. In an additional experiment, the analyte of disodium succinate was added into one of the NIP systems while the other NIP system and the MIP system remained the same. After the addition of disodium succinate, the polymer or hydrogel solid forms in solutions changes to a same red color as that in the MIP system after 50 minutes. These results and observations demonstrate that Au nanoparticles and the chromophore (SP and MC] may be used as a suitable detection method or a device, as the combination of Au nanoparticles and the chromophore (SP and MC] yields a colorimetric readout upon exposure to the analyte of disodium succinate.

The present invention has been described in terms of one or more preferred embodiments, and it should be appreciated that many equivalents, alternatives, variations, and modifications, aside from those expressly stated, are possible and within the scope of the invention.

Each reference identified in the present application is herein incorporated by reference in its entirety.

While present inventive concepts have been described with reference to particular embodiments, those of ordinary skill in the art will appreciate that various substitutions and/or other alterations may be made to the embodiments without departing from the spirit of present inventive concepts. Accordingly, the foregoing description is meant to be exemplary, and does not limit the scope of present inventive concepts.

A number of examples have been described herein. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the present inventive concepts.

What is claimed is:

1. A system for detecting a target in a sample, comprising:
a collection device including:
a collection chamber;
a test chamber;
a passageway between the collection chamber and the test chamber; and
a detection system positioned within the test chamber, the detection system including at least one of a spiropyran, a molecularly imprinted polymer and a nonimprinted polymer.

2. A method for detecting a target in a sample, the method, comprising:
collecting a sample in a collection device including:
a collection chamber;
a test chamber;
a passageway between the collection chamber and the test chamber; and
a detection system positioned within the test chamber, the detection system including at least one of a spiropyran, a molecularly imprinted polymer and a nonimprinted polymer;
contacting the detection system with the sample in the test chamber; and
detecting the presence of a target in the sample with the detection system.

3. The method of claim 2 further comprising the step polymer-gold nanoparticle layer-upon-layer structure.
